(19) United States
(12) Patent Application Publication
(10) Pub. No.: US 2003/0180924 A1
(43) Pub. Date: Sep. 25, 2003

(54) Formulation of certain pyrazolo [3,4,-d] pyrimidines as kinase modulators

(76) Inventor: Robert W. DeSimone, Durham, CT (US)

Correspondence Address: CANTOR COLBURN, LLP, 55 GRIFFIN ROAD SOUTH, BLOOMFIELD, CT, 06002

(21) Appl. No.: 10/393,560

(22) Filed: Mar. 21, 2003

Related U.S. Application Data
(60) Provisional application No. 60/366,567, filed on Mar. 22, 2002.

(51) Int. Cl. C12P 7/00; C12N 9/00

(52) U.S. Cl. 435/183; 435/132

(57) ABSTRACT

Improved formulations of certain pyrazolo[3,4-d]pyrimidines and related compounds, which when appropriately substituted are modulators of kinase activity. These compounds are also useful as probes for the identification of kinases of therapeutic interest.

### CROSS REFERENCE TO RELATED APPLICATIONS

This application claims the benefit of U.S. Provisional Application Serial No. 60/366,567, filed Mar. 22, 2002, which is incorporated by reference herein in its entirety.

### BACKGROUND

This invention relates to an improved formulation of certain pyrazolo[3,4-d]pyrimidines and related compounds, which when appropriately substituted, are modulators of kinase activity. This invention also relates to the use of such compounds as probes for the identification of kinases of therapeutic interest.

One of the central post-translational control elements in eukaryotic signal transduction is the phosphorylation of the hydroxyl moiety of serine, threonine, or tyrosine. The phosphorylation state of a given protein can govern its enzyme activity, protein-protein binding interactions, and cellular distribution. Phosphorylation and dephosphorylation is thus a “chemical switch”, which allows the cell to transmit signals from the plasma membrane to the nucleus and to ultimately control gene expression. Although the exact mechanisms of signal transduction have yet to be elucidated, kinases are thus involved in the control of cell metabolism, growth, differentiation, and apoptosis. These signaling mechanisms affect the onset of cancer, metabolic disorders (for example diabetes), and even infectious diseases. Certain kinases have in fact been implicated in cell proliferation and carcinogenesis. For example, many human cancers are caused by disregulation of a normal protein (e.g., when a proto-oncogene is converted to an oncogene through a gene translocation). Because kinases are key regulators they are ideal drug design targets.

The precise signaling cascades involved in protein kinase-mediated signal transduction have thus far been difficult to elucidate, due in part to the overlapping protein specificities of kinases, their structural and catalytic similarities, and their large number. One approach, set forth in WO 00/42042 to Shokat et al., uses a specific point mutation in a given kinase to create a unique pocket in the substrate binding site of the enzyme. The pocket is designed so as to be absent in any other enzyme in the genome. An inhibitor specific to the engineered enzyme is then designed by attaching a bulky group that fits into the pocket to a known inhibitor. This inhibitor can then be used to study the function of the enzyme, as well as for therapeutic purposes.

### SUMMARY

An improved formulation of certain pyrazolo[3,4-d]pyrimidines comprises a stabilization agent; a solubilization agent; a solvent; and a compound of Formula I

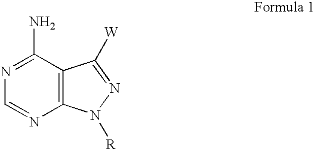

and its active salts and prodrugs, wherein

R is a straight or branched chain C₁-C₇ alkyl, a 5-, 6-, or 7-membered cycloalkyl, or a 5-, 6-, or 7-membered heterocyclic radical which could be unsubstituted or substituted with one or more of hydroxy, nitro, cyano, amino, halogen, C₁-C₇ alkyl, perfluorinated C₁-C₄ alkyl, C₁-C₆ alkoxy, mono- or di(C₁-C₆ alkyl)amino, amino(C₁-C₆ alkyl); and

W is m-phenyoxyphenyl, m-benzyloxyphenyl, m-2,6-dichlorobenzyloxyphenyl, 3-piperonylphenyl, 4-t-butylphenyl, 1-napthoxyylmethyl radical, or a radical having the following structure

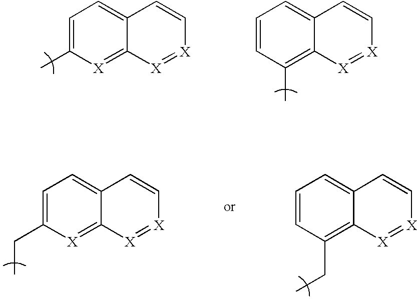

wherein X is CH or N, and which is unsubstituted, mono-, di-, or trisubstituted with one or more hydroxy, nitro, cyano, amino, halogen, C₁-C₇ alkyl, perfluorinated C₁-C₄ alkyl, C₁-C₆ alkoxy, mono- or di(C₁-C₆ alkyl)amino, or amino(C₁-C₆ alkyl) groups. Such formulations are of particular utility for the elucidation of protein kinase-mediated signal transduction.

In another embodiment, a method of inhibiting the phosphorylation of a substrate of a mutant protein kinase by contacting the mutant protein kinase and its substrate with a composition comprising a stabilization agent, a solubilization agent, and a compound of Formula 1.

In another embodiment, an improved pharmaceutical formulation of certain pyrazolo[3,4-d]pyrimidines comprises a stabilization agent; a solubilization agent; a solvent; and a compound of Formula 2:

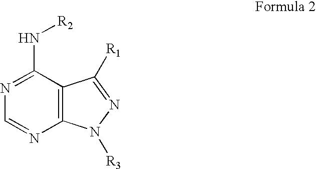

and the pharmaceutically-acceptable salts and prodrugs thereof, wherein

R₁ is methyl, trifluoromethyl, straight or branched chain C₁ to C₇ alkyl, or perfluorinated C₂-C₄ alkyl;

R₂ is hydrogen, straight or branched chain C₁-C₇ alkyl, phenyl which could be unsubstituted, mono-, di- or trisubstituted with one or more of hydroxy, nitro, cyano, amino, halogen, C₁-C₇ alkyl, perfluorinated C₁-C₄ alkyl, C₁-C₆ alkoxy, mono- or di(C₁-C₆ alkyl)amino, amino(C₁-C₆ alkyl), benzyl which could be unsubstituted, mono-, di- or trisubstituted with one or more of hydroxy, nitro, cyano, amino, halogen, C₁-C₆ alkyl, perfluorinated C₁-C₄ alkyl, C₁-C₆ alkoxy, mono- or di(C₁-C₆ alkyl)amino, or amino(C₁-C₆ alkyl), morpholino(C₁-C₄ alkyl), carboxy(C₁-C₃ alkyl), (C₁-C₄ alkoxy)carbonyl(C₁-C₄ alkyl), heterocyclic ring which could be unsubstituted or substituted with one or more of hydroxy, nitro, cyano, amino, halogen, C₁-C₇ alkyl, perfluorinated C₁-C₄ alkyl, C₁-C₆ alkoxy, mono- or di(C₁-C₆ alkyl)amino, amino(C₁-C₆ alkyl); and

R₃ is straight or branched chain C₁-C₇ alkyl, 5, 6, or 7 membered cycloalkyl ring, or 5, 6, or 7 membered heterocyclic ring which could be unsubstituted or substituted with one or more of hydroxy, nitro, cyano, amino, halogen, C₁-C₇ alkyl, perfluorinated C₁-C₄ alkyl, C₁-C₆ alkoxy, mono- or di(C₁-C₆ alkyl)amino, amino(C₁-C₆ alkyl).

In another embodiment, a pharmaceutical composition comprises a therapeutically effective amount of a compound of Formula 1 or 2 and a stabilization agent; a solubilization agent; and a solvent.

In still another embodiment, a method of treating a kinase-implicated disorder in a mammal comprises administration to the mammal of therapeutically effective amount of a compound of Formula 1 or 2 and a stabilization agent; a solubilization agent; and a solvent.

### DETAILED DESCRIPTION

The following definitions are used herein.

When any variable occurs more than one time in Formula 1 or 2, its definition on each occurrence is independent of its definition at every other occurrence.

By “C₁-C₆ alkyl” and “C₁-C₇ alkyl” is meant straight or branched chain alkyl groups or cycloalkyl groups having 1-6 and 1-7 carbon atoms respectively, such as, for example, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl. Preferred C₁-C₆ alkyl and C₁-C₇ alkyl groups are methyl, ethyl, propyl, butyl, cyclopropyl, and cyclopropylmethyl, cyclohexyl, cycloheptyl, norbornyl, and the like.

By “C₁-C₆ alkoxy” is meant an alkyl group of indicated number of carbon atoms attached through an oxygen bridge such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy. Preferred alkoxy groups herein are C₁-C₄ alkoxy groups.

The term “halogen” includes fluorine, chlorine, bromine, and iodine.

By “heterocyclic” is meant heteroaryl or heterocycloalkyl. By “heteroaryl” is meant, but is not limited to, systems (as numbered from the linkage position assigned priority 1), such as 2-pyridyl, 3-pyridyl, 4-pyridyl, 2,3-pyrazinyl, 3,4-pyrazinyl, 2,4-pyrimidinyl, 3,5-pyrimidinyl, 2,3-pyrazolinyl, 2,4-imidazolinyl, isoxazolinyl, oxazolinyl, thiazolinyl, thiadiazolinyl, tetrazolyl, thienyl, benzothiophenyl, furanyl, benzofuranyl, benzoimidazolinyl, indolinyl, and pyrrolyl.

By “heterocycloalkyl” is meant an aliphatic ring containing at least 1 carbon atom in addition to 1-3 heteroatoms independently selected from oxygen, sulfur, or nitrogen, such as, but not limited to, morpholinyl, thiomorpholinyl, piperidinyl, and pyrrolidinyl.

If the compounds of Formula 1 or 2 have asymmetric centers, then Formula 1 and 2 includes all of the optical isomers and mixtures thereof. In addition, compounds with carbon-carbon double bonds may occur in Z- and E-forms, with all isomeric forms of the compounds being included. These compounds can be, for example, racemates or optically active forms. In these situations, the single enantiomers, i.e., optically active forms can be obtained by asymmetric synthesis or by resolution of the racemates. Resolution of the racemates can be accomplished, for example, by conventional methods such as crystallization in the presence of a resolving agent, or chromatography, using, for example a chiral HPLC column. Where a compound exists in various tautomeric forms, the invention is not limited to any one of the specific tautormers, and includes all tautomeric forms of the compound.

Representative compounds of the present invention, which are encompassed by Formula 1 or 2, include, but are not limited to their pharmaceutically acceptable acid addition salts. Non-toxic “pharmaceutically acceptable salts” include, but are not limited to salts with inorganic acids, such as hydrochlorate, phosphate, diphosphate, hydrobromate, sulfate, sulfinate, or nitrate salts; or salts with an organic acid, such as malate, maleate, fumarate, tartrate, succinate, citrate, acetate, lactate, methanesulfonate, p-toluenesulfonate, 2-hydroxyethylsulfonate, benzoate, salicylate, stearate, and alkanoate such as acetate, HOOC—(CH₂)n-ACOOH where n is 0-4, and the like salts. Similarly, pharmaceutically acceptable cations include, but are not limited to sodium, potassium, calcium, aluminum, lithium, and ammonium.

In addition, if the compound of the invention is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts encompassed by Formula 1 or 2.

The present invention also encompasses the prodrugs of the compounds of Formula 1 or 2, for example acylated prodrugs of the compounds of Formulas 1 and 2. Those skilled in the art will recognize various synthetic methodologies that may be used to prepare non-toxic pharmaceutically acceptable acylated and other prodrugs of the compounds encompassed by Formulas 1 and 2.

Methods for obtaining the compounds described herein are known to those of ordinary skill in the art, suitable procedures being described, for example, in the references cited herein.

In one embodiment, an improved formulation for elucidation of protein kinase-mediated signal transduction comprises a stabilization agent; a solubilization agent; a solvent; and a compound of Formula 1. Such formulations are of particular utility for in vivo administration to a mammal comprising an engineered kinase that specifically binds the compound of Formula 1, as described in the aforementioned WO 00/42042. They may also be used to elucidate which kinases may be involved in disease; these kinase can then become the subject to efforts to design or discover more traditional specific inhibitors of their wild-type forms, which may prove to be valuable in treating the kinase-related disease or disorder.

In another embodiment, an improved pharmaceutical formulation of certain pyrazolo[3,4-d]pyrimidines comprises a stabilization agent; a solubilization agent; a solvent; and a therapeutically effective amount of a compound of Formula 2. These compounds are hypothesized to be inhibitors of at least one wild-type protein kinase, and may accordingly be used for the alleviation or prevention of conditions associated with such kinase.

In the compositions comprising a compound of Formula 1 or Formula 2 (or a combination of the two), suitable stabilization agents are antioxidants, for example butylated hydroxy anisole, butylated hydroxy toluene, tocopherolacetate, polyethylene glycol 1000, succinate, ascorbic acid, ascorbyl palmitate, butylated hydroxyanisole, a mixture of 2- and 3-tertiary-butyl-4-hydroxyanisole, butylated hydroxytoluene, sodium isoascorbate, dihydroguaretic acid, potassium sorbate, sodium ascorbate, sodium bisulfate, sodium metabisulfate, sorbic acid, potassium ascorbate, Vitamin E (alpha-tocopherol), Vitamin C, folic acid, Vitamin B₆, Vitamin B₁₂, 4-chloro-2-,6-ditertiary butylphenol, propylgallate or a mixture comprising one of the foregoing antioxidants. A preferred stabilization agent is Vitamin E. The amount of stabilization agent is selected so as to provide effective stabilization during administration of the compound of Formula 1 or 2, and thus will vary depending on the compound and formulation of the composition. In general, at least about 10%, preferably at least about 5%, and most preferably at least about 3% (weight percent) of the stabilization agent is present, based on the total weight of the composition. Less than about 10%, preferably less than about 5%, and most preferably less than about 3% (weight percent) of the stabilization agent is present, based on weight percent of the stabilizing agent in water.

A solubilization agent is also present, to increase availability of the compound of Formula 1 or 2 in vivo. A suitable solubilization agent will increase the solubility of a compound of Formula 1 or 2 in water to greater than or equal to 0.1 mg/mL of water, preferably greater than or equal to 0.5 mg/mL of water, more preferably greater than or equal to 1.0 mg/mL of water. Suitable solubilization agents include non-ionic detergents such as n-octylglucoside, n-dodecylglucoside, n-dodecylmaltoside, octanoyl-N-methylglucamide, decanoyl-N-methylglucamide, octylphenol ethylene oxide condensate (e.g., Tritong X-100 or Triton® X-114, commercially available from Aldrich Chemical Company, Inc.), polidocanol (e.g., Thesit®, commercially available from Roche), isotridecypoly(ethylene glycol ether)ₙ, 3-[(3-cholamidopropyl)dimethylammonio]-1-propane sulfonate (CHAPS), 3-[(3-cholamidopropyl)dimethylammonio]-2-hydroxy-1-propane sulfonate (CHAPSO), or N-dodecyl-N,N-dimethyl-3-ammonio-1-propane sulfonate. Other solubilizing agents include polyoxyethylene sorbitan monostearate (e.g. TWEEN-80® or TWEEN-20®, commercially available from Aldrich Chemical Company), a non-ionic poloxamer surfactant (e.g. PLURONIC F108®, commercially available from BASF Corp.), a glycerol ester or diester of a fractionated fatty acid having a chain length of 8 to 10 carbons (e.g., MIGLYOL 829® or MIGLYOL 840®, commercially available from Huls America, Inc., Piscataway, N.J.). Other useful solubilization agents include propylene glycol, glycerin, glycerol, or conventional polyethylene glycols such as polyethylene glycol (PEG) 200, 300, 400, and the like. Mixtures comprising at least one of the foregoing solubilization agents may also be used.

The amount of solubilization agent is selected so as to provide effective solubilization during administration of the compound of Formula 1 or 2, and thus will vary depending on the compound and formulation of the composition. At least about 70%, preferably at least about 60%, and most preferably at least about 50% (volume) of the solubilization agent is present, based on the total weight of the composition. Less than about 70%, preferably less than about 60%, and most preferably less than about 50% (volume) of the solubilization agent is present, based on % volume in water.

The stabilization and solubilization agents may be provided to the composition separately, or together, i.e., covalently bound. Such combined stabilization/solubilization agents are known, and include, for example d-alpha-tocopherol polyethyleneglycol 1000 succinate, which is commercially available under the trade name “Vitamin E TPGS, NF Grade” from Eastman Chemicals. In an advantageous feature, the effective concentrations of such combined agents are at least about 0.01%, preferably at least about 0.05%, and most preferably at least about 1% (weight percent) of the combined agent, based on the total weight of the composition. Less than about 10%, preferably less than about 5%, and most preferably less than about 3% (weight percent) of the combined agent is generally used, based on weight percent in water. Of course, larger amounts of a combined stabilizing agent/solubilization agent may be used where appropriate.

The preferred solvent is water, although mixtures of water with small amounts of organic solvents may also be used alone or in combination, for example ethanol, mineral oil, vegetable oil, or dimethyl sulfoxide (DMSO). When present, the amount of organic solvent is less than about 10 volume percent, preferably less than about 2 volume percent of the total composition.

The amount of the compound of Formula 1 or 2 (or a combination thereof) in the composition will depend on factors such as activity, stability, method of administration, purpose of administration (i.e., determination of a dose-response effect) and the like. In general, the compound of Formula 1 or 2 is present in an amount from about 0.5 to about 10 mg/mL.

The present invention also pertains to methods of inhibiting the binding of a protein substrate to its corresponding kinase, which methods involve contacting an improved composition of the present invention with cells expressing the kinase, wherein the compound is present at a concentration sufficient to inhibit substrate protein binding to the kinase in vitro. This method includes inhibiting the binding of substrate protein to the corresponding kinase in vivo, e.g., in an animal that expresses the kinase naturally, or that is engineered to express the kinase. The amount of a compound of Formula 1 or 2, its salt, or prodrugs thereof that would be sufficient to inhibit the binding of substrate protein to the corresponding kinase in vitro may be readily determined via a kinase binding assay as is known in the art.

Suitable kinases include but are not limited to tyrosine kinases and serine/threonine kinases, which may be classified as including the AGC group (cyclic nucleotide regulated family) of protein kinases, which includes the cyclic nucleotide regulated protein kinase family (e.g., PKA and PKG), the diacylglycerol-activated/phospholipid-dependent family protein kinase C family (e.g. PKC), the PKA and PKC-related family (e.g. RAC and Akt), the kinases that phosphorylate G protein-coupled receptors family, the budding yeast AGC-related protein kinase family, the kinases that phosphorylate ribosomal protein S6 family, the budding yeast DBF2/20 family, the flowering plant PVPK1 protein kinase homolog family, and other AGC related kinase families.

The CaMK (calcium calmodulin dependent) group of protein kinases includes kinases regulated by Ca²⁺/CaM and close relatives family, the KIN1/SNF1/Nim1 family, and other related CaMK related kinase families. The CMGC group (named because it includes the cyclin-dependent kinases) includes the cyclin-dependent kinases (e.g. CDKs) and close relatives family, the ERK (e.g. MAP) kinase family, the glycogen synthase 3 (e.g. GSK3) family, the casein kinase II family, the Clk family and other CMGC kinases.

The PTK group of protein kinases includes protein-tyrosine kinases that may be nonmembrane-spanning or membrane-spanning tyrosine kinases. The PTK group of protein kinases includes the Src family, the Tek/Atk family, the Csk family, the Fes (Fps) family, the Abl family, the Syk/ZAP70 family, the Ttk2/Jak1 family, the Ack family, the focal adhesion kinase (Fak) family, the epidermal growth factor receptor family, the Eph/Elk/Eck receptor family, the Axl family, the Tie/Tek family, the platelet-derived growth factor receptor family, the fibroblast growth factor receptor family, the insulin receptor family, the LTK/ALK family, the Ros/Sevenless family, the Trk/Ror family, the DDR/TKT family, the hepatocyte growth factor receptor family, the nematode Kin15/16 family, the Tec family which includes such kinases as Btk and Itk, and other PTK kinase families.

The OPK group (other protein kinases) includes the Polo family, the MEK/STE7 family, the PAK/STE20 family, the MEKK/STE11 family, the NimA family, the wee1/mik1 family, the kinases involved in transcriptional control family, the Raf family, the Activin/TGFb receptor family, the flowering plant putative receptor kinases and close relatives family, the PSK/PTK leucine zipper domain family, the casein kinase I family, the PKN prokaryotic protein kinase family and other OPK protein kinase families. A large number of kinases are found in G. Hardie et al., Protein Kinase Facts Book 0-12-324719-5 (1995).

The present invention also pertains to in vivo methods for altering the signal-transducing activity of a given kinase, particularly phosphorylation, the method comprising exposing cells expressing such kinases to the improved composition described above, comprising an effective amount of a compound of Formula 1 or 2. This method includes altering the signal-transducing activity of the kinase in an animal given an amount of a compound of Formula 1 or 2 that would be sufficient to alter the signal-transducing activity of kinase in vitro. The amount of a compound of Formula 1 or 2 that would be sufficient to alter the signal-transducing activity of the kinase be determined via a kinase signal transduction assay, such as is known in the art.

In still another embodiment, a method of treating a kinase-implicated disorder in a mammal comprises administration to the mammal of therapeutically effective amount of a compound of Formula 1 or 2 and a stabilization agent; a solubilization agent; and a solvent. Kinases are implicated in a large variety of diseases, as certain mutations in protein kinases can lead to activation of pathways causing, for example, the production of tumors, while other mutations in protein kinases block pathways and prevent a response. Diseases linked to mutations in protein kinases are listed in the KinMutBase database (http://www.uta.fi/imt/bioinfo/KinMutBase/) (Stenberg et al., Nucleic Acids Research, Vol. 28, pp. 369-372, 2000). Diseases caused by protein kinase mutations include X-linked agammaglobulinemia (XLA), and non-insulin dependent diabetes mellitus (NIDDM), and severe combined immunodeficiency (SCID). Mutations related to tumor development have been liked to such diseases as Hirschprung's disease, multiple endocrine neoplasia type 2 (MEN2) a and b, medullary thyroid carcinoma (FMTC), papillary renal carcinoma (HPRC), and Peutz-Jeghers syndrome.

Mutations in growth factor receptor kinases are linked to diseases such as mastocytosis, systemic mast cell disease, piebaldism, hypochondroplasia, thanatophoric dysplasia, and skeletal dysplasia. Other protein kinase-linked diseases include Coffin-Lowry syndrome, congenital insensitivity to pain with anhidrosis (CIPA), hypertension, vascular dysplasia, errors in vascular morphogenesis, and X-linked mental retardation. Mutations in protein kinases have also been linked to neurodegenerative diseases such as amyotrophic lateral sclerosis (ALS) and Alzheimer's disease (AD).

Other diseases associated with protein kinases are Gaucher disease, hypochromic anemia, granulomatous disease, ataxia-telangiectasia, familial hypercholesterolemia, certain types of muscular dystrophy such as Driefuss-Emory type, cystic fibrosis, type 1 hyperlipoproteinemia, Treacher Collins Franceschetti syndrome 1, Tay-Sachs disease, type 1 neurofibromatosis, adenomatous polyposis of the colon, X-linked ichthyosis and Beckwith-Weidemann Syndrome.

Altered PKA (cyclic AMP-dependent protein kinase) expression is implicated in a variety of disorders and diseases including cancer, thyroid disorders, diabetes, atherosclerosis, and cardiovascular disease. Altered MAP (mitogen-activated protein) kinase expression is implicated in a variety of disease conditions including cancer, inflammation, immune disorders, and disorders affecting growth and development. RTKs (receptor tyrosine kinases), CDKs and STKs (serine/threonine kinases) have all been implicated in a host of pathogenic conditions including, significantly, large number of diverse cancers. Others pathogenic conditions which have been associated with PTKs include, without limitation, psoriasis, hepatic cirrhosis, diabetes, atherosclerosis, angiogenesis, restinosis, ocular diseases, rheumatoid arthritis and other inflammatory disorders, autoimmune disease and a variety of renal disorders.

Preferably, the conditions, diseases and/or disorders that can be affected using compounds and compositions according to the invention include, but are not limited to, psoriasis, cancer (for example, chronic myelogenous leukemia, gastrointestinal stromal tumors, non-small cell lung cancer, breast cancer, ovarian cancer, recurrent ovarian cancer, prostate cancer such as hormonal refractory prostate cancer, kidney cancer, head and neck cancer, or colorectal cancer), immunoregulation (graft rejection), atherosclerosis, rheumatoid arthritis, Parkinson's disease, Alzheimer's disease, diabetes (for example insulin resistance or diabetic retinopathy), septic shock, and the like.

The following examples are provided to describe and illustrate the present invention. As such, they should not be construed to limit the scope of the invention. Those in the art will well appreciate that many other embodiments also fall within the scope of the invention, as it is described hereinabove and in the claims.

### EXAMPLES

In these examples, formulations were prepared from formula 1 wherein R is a tertiary-butyl group and W is a naphthyl group (X being carbon atoms) (“1-Na-PP1”), or formula 1 wherein R is a tertiary butyl group and W is a methyl napthyl group (X being carbon atoms) (“1-NM-PP1”) as shown below.

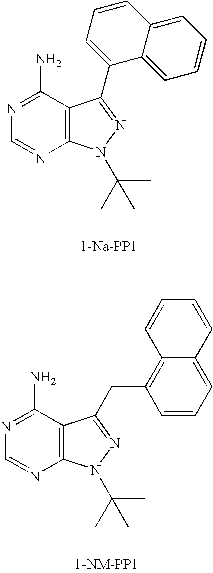

Comparative formulations comprised the free base of each compound at the indicated concentration. Improved formulations of the present invention comprises the hydrochloric acid salt of each compound and Vitamin E-TPGS prepared as indicated below (the procedures were similar for NM-PP1).

Preparation of the Hydrochloride Salt of Na-PP1.

A quantity of 0.6 g (1.89 mmole) of 1-tert-butyl-3-naphthalen-1-yl-1H-pyrazolo[3,4-d]pyrimidin-4-ylamine (Na-PP1) is dissolved in 30 mL of anhydrous ethyl acetate. To the solution, 8 mL of 2M HCl in diethyl ether is added at room temperature. The resulting mixture is allowed to stand at room temperature until the salt crystallizes out of solution. The salt is washed with diethyl ether and allowed to dry, to afford 0.650 mg (0.184 mmole) of 1-tert-butyl-3-naphthalen-1-yl-1H-pyrazolo[3,4-d]pyrimidin-4-ylamine-HCl (99% yield).

Formulation of Na-PP1-HCl Vitamin E TPGS.

A quantity of 5 g of d-alpha-tocopherol polyethyleneglycol 1000 succinate is dissolved in 20 mL of distilled deionzed water and warmed at 40° C. for 4 hrs to provide a stock solution. A quantity of 0.5 mL of the stock d-alpha-tocopherol polyethyleneglycol 1000 succinate solution is added to 5 mg of Na-PP1-HCl and warmed until all material is in solution. The resulting mixture is diluted with 4.5 mL of distilled deionzed water to afford a 1-mg/mL formulation of Na-PP1-HCl in Vitamin E TPGS.

Bioavailability Testing.

Compositions comprising the free base of 1-Na-PP1 and 1-NM-PP1 (Control) and the hydrochloride salt of 1-Na-PP1 and 1-NM-PP1 formulated with Vitamin E TPGS are administered to male CD-1 mice intraperitoneally (IP) and intravenously (IV). Whole blood samples were collected from three animals per time point and prior via cardiac puncture into EDTA tubes and placed on wet ice. Samples were analyzed after drying and predissolution in 0.1:20:80 formic acid:methanol:water by liquid chromatography/mass spectrometry using an Xterra C182.5 micromenter particle size analytical column (15 mm×2.1 mm). Results are shown in Table 1 below.

TABLE 1
IV Treatment

| | | 1-Na-PP1- | | 1-NM-PP1- |
|---|---|---|---|---|
| | 1-Na-PP1 | HCl, | 1-NM-PP1 | HCl, |
| | (Control) | Formulated | (Control) | Formulated |
| Dosage, mg/Kg | 0.269 | 2 | 0.793 | 2 |
| Parameter, Units | | | | |
| AUC, ng*Hours/mL | 32.14 | 433 | 37.08 | 463 |
| T_{½}, Hours | 0.46 | 3.27 | 0.39 | 0.83 |
| CL, mL/kg/min | 133.2 | 76 | 129.5 | 72 |
| Vdss, L/Kg | 5.3 | 3.7 | 4.1 | 2.5 |
| AUC/Dose, | 0.12 | 219 | 0.13 | 231 |
| ng*Hours/mL/μg/kg | | | | |

IP Treatment

| | | 1-Na-PP1- | | 1-NM-PP1- |
|---|---|---|---|---|
| | 1-Na-PP1 | HCl, | 1-NM-PP1 | HCl, |
| | (Control) | Formulated | (Control) | Formulated |
| Dosage, mg/Kg | 0.793 | 20 | 0.793 | 20 |
| Parameter, Units | | | | |
| Cmax, ng/mL | 55 | 5850 | 90.6 | 3452 |
| Tmax, hours | 0.083 | 0.08 | 0.083 | 0.08 |
| AUC, ng*Hours/ml | 99.37 | 2787 | 134.84 | 2373 |
| T_{½}, hours | 1.51 | 10.9 | 1.42 | 4.7 |
| AUC/Dose, | 0.13 | 139 | 0.16 | 118 |
| ng*Hours/mL/μg/kg | | | | |
| Bioavailability, % | 100 | 63% | 100 | 52% |

Animals were euthanized after 0.8, 1, 8, and 48 hours and various tissues analyzed for the presence of the test compounds. Table 2 shows the measured tissue distribution of the formulated hydrochloride salt of Na-PP1 administered at 20 mg/Kg.

TABLE 2
IP dosing (n = 4)

| Time (hr) | Brain (ng/g) | Testis (ng/g) |
|---|---|---|
| 0.08 | 750 | 2435 |
| 1 | 911 | 2152 |
| 8 | 71 | 659 |
| 48 | 26.2 | 97 |

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended embodiments.

All cited references are incorporated herein in their entirety.

What is claimed is:

1. An improved formulation comprising:
a stabilization agent; a solubilizafion agent; a solvent; and a compound of Formula 1 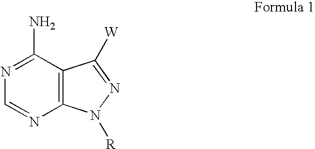
and its pharmaceutically active salts and prodrugs, wherein
R is a straight or branched chain C₁-C₇ alkyl, a 5-, 6-, or 7-membered cycloalkyl, or a 5-, 6-, or 7-membered heterocyclic radical which could be unsubstituted or substituted with one or more of hydroxy, nitro, cyano, amino, halogen, C₁-C₇ alkyl, perfluorinated C₁-C₄ alkyl, C₁-C₆ alkoxy, mono- or di(C₁-C₆ alkyl)amino, amino(C₁-C₆ alkyl); and
W is m-phenyoxyphenyl, m-benzyloxyphenyl, m-2,6-dichlorobenzyloxyphenyl, 3-piperonylphenyl, 4-t-butylphenyl, 1-napthoxyylmethyl radical, or a radical having the following structure 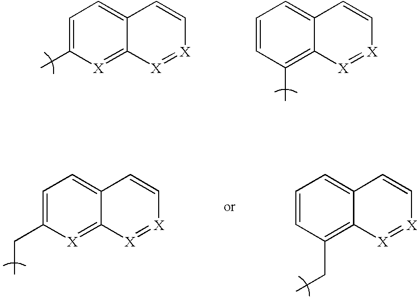
wherein x is CH or N, and which is unsubstituted, mono-, di-, or trisubstituted with one or more hydroxy, nitro, cyano, amino, halogen, C₁-C₇ alkyl, perfluorinated C₁-C₄ alkyl, C₁-C₆ alkoxy, mono- or di(C₁-C₆ alkyl)amino, or amino(C₁-C₆ alkyl) groups.

2. The formulation of claim 1, wherein the stabilization agent and the solubilization agent is d-alpha-tocopherol polyethyleneglycol 1000 succinate.

3. A method of inhibiting the phosphorylation of a substrate of a mutant protein kinase by contacting the mutant protein kinase and its substrate with the formulation of claim 1 or claim 2.

4. An improved pharmaceutical formulation, comprising:
a stabilization agent; a solubilization agent; a solvent; and a compound of Formula 2 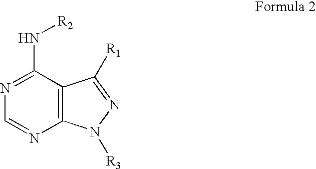
and the pharmaceutically-acceptable salts and prodrugs thereof, wherein
R₁ is methyl, trifluoromethyl, straight or branched chain C₁ to C₇ alkyl, or perfluorinated C₂-C₄ alkyl;
R₂ is hydrogen, straight or branched chain C₁-C₇ alkyl, phenyl which could be unsubstituted, mono-, di- or trisubstituted with one or more of hydroxy, nitro, cyano, amino, halogen, C₁-C₇ alkyl, perfluorinated C₁-C₄ alkyl, C₁-C₆ alkoxy, mono- or di(C₁-C₆ alkyl)amino, amino(C₁-C₆ alkyl), benzyl which could be unsubstituted, mono-, di- or trisubstituted with one or more of hydroxy, nitro, cyano, amino, halogen, C₁-C₆ alkyl, perfluorinated C₁-C₄ alkyl, C₁-C₆ alkoxy, mono- or di(C₁-C₆ alkyl)amino, or amino(C₁-C₆ alkyl), morpholino(C₁-C₄ alkyl), carboxy(C₁-C₃ alkyl), (C₁-C₄ alkoxy)carbonyl(C₁-C₄ alkyl), heterocyclic ring which could be unsubstituted or substituted with one or more of hydroxy, nitro, cyano, amino, halogen, C₁-C₇ alkyl, perfluorinated C₁-C₄ alkyl, C₁-C₆ alkoxy, mono- or di(C₁-C₆ alkyl)amino, amino(C₁-C₆ alkyl); and
R₃ is straight or branched chain C₁-C₇ alkyl, 5, 6, or 7 membered cycloalkyl ring, or 5, 6, or 7 membered heterocyclic ring which could be unsubstituted or substituted with one or more of hydroxy, nitro, cyano, amino, halogen, C₁-C₇ alkyl, perfluorinated C₁-C₄ alkyl, C₁-C₆ alkoxy, mono- or di(C₁-C₆ alkyl)amino, amino(C₁-C₆ alkyl).

5. The formulation of claim 4, wherein the stabilization agent and the solubilization agent is d-alpha-tocopherol polyethyleneglycol 1000 succinate.

6. A pharmaceutical composition comprising a therapeutically effective amount of a compound of Formula 1, Formula 2, or a combination of Formula 1 and Formula 2; a stabilization agent; a solubilization agent; and a solvent.

7. The composition of claim 6, wherein the stabilization agent and the solubilization agent is d-alpha-tocopherol polyethyleneglycol 1000 succinate.

8. A method of treating a kinase-implicated disorder in a mammal, comprising administration to the mammal of a therapeutically effective amount of a compound of Formula 1, Formula 2, or a combination of Formula 1 and Formula 2; a stabilization agent; a solubilization agent; and a solvent.
